(19) United States
(12) Patent Application Publication
(10) Pub. No.: US 2006/0014980 A1
(43) Pub. Date: Jan. 19, 2006

(54) Catalyst for alpha, beta-unsaturated carboxylic acid production, process for producing the same, and process for producing alpha, beta-unsaturated carboxylic acid

(75) Inventors: Seiichi Kawato, Hiroshima (JP); Akio Takeda, Hiroshima (JP); Wataru Ninomiya, Hiroshima (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Mitsubishi Rayon co., Ltd., Tokyo (JP)

(21) Appl. No.: 10/531,461

(22) PCT Filed: Oct. 27, 2003
(86) PCT No.: PCT/JP03/13710

§ 371 Date: Apr. 15, 2005

(30) Foreign Application Priority Data
Oct. 28, 2002 (JP) 2002-312492

(51) Int. Cl. C07C 51/16

(52) U.S. Cl. 562/545

(57) ABSTRACT

A catalyst for preparation of an α,β-unsaturated carboxylic acid in which a precious metal is supported on activated carbon having a specific surface area of 100 m²/g or more and 1300 m²/g or less is a catalyst by which the α,β-unsaturated carboxylic acid can be prepared from an olefin or α,β-unsaturated aldehyde in a high yield. This catalyst can be prepared by selecting activated carbon having a specific surface area of 100 m²/g or more and 1300 m²/g or less and loading a precious metal on the activated carbon.

### TECHNICAL FIELD

The present invention relates to a catalyst for oxidizing an olefin or α,β-unsaturated aldehyde with molecular oxygen in a liquid phase to prepare an α,β-unsaturated carboxylic acid, a preparation method of the catalyst, and a preparation method of the α,β-unsaturated carboxylic acid.

### BACKGROUND ART

A catalyst for oxidizing an olefin or α,β-unsaturated aldehyde with molecular oxygen in a liquid phase to obtain an α,β-unsaturated carboxylic acid has heretofore been intensively researched. For example, a catalyst in which gold is supported on a carrier is proposed in Japanese Patent Application Laid-Open No. 2001-172222, and a catalyst in which palladium is supported on a carrier is proposed in Japanese Patent Application Laid-Open Nos. 60-155148, 60-139341, and 56-59722. Activated carbon is described as one example of carriers for supporting precious metals in these documents, but there is not any description concerning a surface area of the activated carbon.

<Prior Document List>

Japanese Patent Application Laid-Open No. 2001-172222
Japanese Patent Application Laid-Open No. 60-155148
Japanese Patent Application Laid-Open No. 60-139341
Japanese Patent Application Laid-Open No. 56-59722

### DISCLOSURE OF THE INVENTION

As a result of preparation of acrylic acid from propylene by the use of a precious metal-supported catalyst in accordance with methods described in examples of the above-described documents, the present inventor has found that many polymers or oligomers are incidentally produced in addition to by-products (acetaldehyde, acetone, acrolein, acetic acid, carbon dioxide) described in the above-described documents. It has been revealed that these polymers or oligomers are not trapped and an actual yield of acrylic acids including these is lower than a value described in the examples of the above-described documents. Therefore, the yield of the preparation method of an α,β-unsaturated carboxylic acid has not been sufficient yet, and there has been a demand for a catalyst by which the α,β-unsaturated carboxylic acid can be prepared with a higher yield.

Therefore, an object of the present invention is to provide a catalyst for preparation of an α,β-unsaturated carboxylic acid from an olefin or α,β-unsaturated aldehyde in a high yield, a preparation method of the catalyst, and a preparation method of the α,β-unsaturated carboxylic acid in a high yield.

According to the present invention, there is provided a catalyst for preparation of an α,β-unsaturated carboxylic acid, for oxidizing an olefin or α,β-unsaturated aldehyde with molecular oxygen in a liquid phase to prepare the α,β-unsaturated carboxylic acid, wherein a precious metal is supported on activated carbon having a specific surface area of 100 m²/g or more and 1300 m²/g or less.

Moreover, according to the present invention, there is provided a preparation method of the above-described catalyst for preparation of an α,β-unsaturated carboxylic acid, comprising selecting activated carbon having a specific surface area of 100 m²/g or more and 1300 m²/g or less and loading the precious metal on the activated carbon.

Furthermore, according to the present invention, there is provided a preparation method of an α,β-unsaturated carboxylic acid, comprising a step of carrying out a reaction in which an olefin or α,β-unsaturated aldehyde is oxidized with molecular oxygen in a liquid phase in the presence of the above-described catalyst for preparation of an α,β-unsaturated carboxylic acid to prepare the α,β-unsaturated carboxylic acid.

By the catalyst for the preparation of the α,β-unsaturated carboxylic acid of the present invention, the olefin or α,β-unsaturated aldehyde can be oxidized with molecular oxygen in the liquid phase to prepare the α,β-unsaturated carboxylic acid with the high yield. The catalyst of the present invention is preferable for liquid-phase oxidation in which acrylic acid is prepared from propylene or acrolein or methacrylic acid is prepared from isobutylene or methacrolein.

Moreover, according to the preparation method of the catalyst for the preparation of the α,β-unsaturated carboxylic acid of the present invention, the olefin or α,β-unsaturated aldehyde can be oxidized with molecular oxygen in the liquid phase to prepare the α,β-unsaturated carboxylic acid with the high yield.

Furthermore, according to the preparation method of the α,β-unsaturated carboxylic acid, the α,β-unsaturated carboxylic acid can be prepared with the high yield.

### BEST MODE FOR CARRYING OUT THE INVENTION

The present invention relates to a catalyst for oxidizing an olefin or α,β-unsaturated aldehyde with molecular oxygen in a liquid phase to prepare an α,β-unsaturated carboxylic acid (also hereinafter referred to simply as liquid-phase oxidation), and a precious metal is supported on activated carbon having a specific surface area of 100 m²/g or more and 1300 m²/g or less to constitute the catalyst for the preparation of the α,β-unsaturated carboxylic acid. In accordance with the catalyst for the preparation of the α,β-unsaturated carboxylic acid of the present invention, an amount of by-products produced during oxidation of the olefin or α,β-unsaturated aldehyde with molecular oxygen in the liquid phase to prepare the α,β-unsaturated carboxylic acid is small, and it is possible to prepare the α,β-unsaturated carboxylic acid with the high yield.

The specific surface area of the activated carbon described above is measured in a state before loading the precious metal by a multi-point BET analysis. The specific surface area can be measured with an automatic surface area analyzer such as TriStar 3000 (trade name) made by Shimadzu Corp.

The specific surface area of the activated carbon for use in the present invention is 100 m²/g or more, and preferably 300 m²/g or more. The specific surface area of the activated carbon for use in the present invention is 1300 m²/g or less, preferably 1000 m²/g or less, more preferably 800 m²/g or less. When the specific surface area is larger than 1300 m²/μg, activity of the catalyst tends to decrease. When the specific surface area is smaller than 100 m²/g, a produced amount of by-products tends to increase. In either case, the yield of the α,β-unsaturated carboxylic acid decreases.

The activated carbon for use in the present invention is not especially limited by a raw material, form, presence/absence of activation, and activation method, as long as the above-described conditions of the specific surface area are satisfied. Examples of raw materials of the activated carbon include woods, coconut shells, coals and synthetic resins. Examples of the form of the activated carbon include powdered form, crushed form, granular form, pelletized form and fibrous form. Examples of an activation method of the activated carbon include steam activation, carbon dioxide activation, zinc chloride activation, phosphoric acid activation and alkali activation.

It is to be noted that the examples of a control method of the specific surface area of the activated carbon include a method of controlling an activation temperature and/or activation time during the activation of the raw material. In general, when the activation temperature increases, the specific surface area of the activated carbon tends to increase. When the activation time is longer, the specific surface area of the activated carbon tends to increase.

The precious metal supported on the activated carbon described above is one or more selected from a group consisting of palladium, platinum, rhodium, ruthenium, iridium, gold, silver, and osmium, and is, above all, preferably one or more selected from a group consisting of palladium, platinum, rhodium, ruthenium, iridium, and gold, and palladium is especially preferable.

An amount of loading of the above-described precious metal is usually in a range of 0.1 to 40 wt % with respect to the activated carbon before the loading. The amount of loading of the precious metal with respect to the activated carbon before the loading is preferably 1 wt % or more, more preferably 2 wt % or more, especially preferably 4 wt % or more. The amount of loading of the precious metal with respect to the activated carbon before the loading is preferably 30 wt % or less, more preferably 20 wt % or less, especially preferably 15 wt % or less.

The catalyst for the preparation of the α,β-unsaturated carboxylic acid of the present invention is preferable as a catalyst for preparation of the α,β-unsaturated carboxylic acid from the olefin or α,β-unsaturated aldehyde. Above all, the catalyst is especially preferable as a catalyst for the preparation of acrylic acid from propylene or acrolein, or a catalyst for the preparation of methacrylic acid from isobutylene or methacrolein.

The preparation method of the above-described catalyst for the preparation of the α,β-unsaturated carboxylic acid of the present invention is not especially limited, and it is possible to use a method in which the activated carbon having a specific surface area of 100 m² μg or more and 1300 m²/g or less is selected, and the precious metal is loaded on the activated carbon. Preferably, the activated carbon having a specific surface area of 100 m²/g or more and 1000 m²/g or less is selected, and the precious metal is loaded on the activated carbon.

More concretely, a precious metal compound corresponding to the precious metal to be loaded can be reduced by a reducing agent in the presence of the activated carbon to prepare the catalyst. For example, the catalyst can be prepared by a liquid-phase reduction in which the reducing agent is added to a solution of the precious metal compound in which the activated carbon is dispersed, and the precious metal is reduced and loaded on the activated carbon. The catalyst can also be prepared by a gas-phase reduction in which the activated carbon impregnated with the solution of the precious metal compound is dried to support the precious metal compound by the activated carbon, and thereafter the supported precious metal compound is reduced in a reductive atmosphere. Above all, the liquid-phase reduction is preferable. The preparation method of the catalyst by the liquid-phase reduction will hereinafter be described.

The precious metal compound is not especially limited. However, a preferable example thereof is a chloride, oxide, acetate, nitrate, sulfate, tetra-ammine complex or acetylacetonate complex of the precious metal; a more preferable example thereof is a chloride, oxide, acetate, nitrate or sulfate of the precious metal; and an especially preferable example thereof is a chloride, acetate or nitrate of the precious metal.

As the solvent in which the precious metal is dissolved, there can be used water, an alcohol, ketone, organic acid, hydrocarbon, or mixed solvent of two or more selected from a group consisting of them. The solvent is appropriately selected by solubility of the precious metal compound or the reducing agent or dispersibility of the carrier.

The activated carbon and precious metal compound are added to the solvent in a desired order or simultaneously to prepare a precious metal compound solution in which the activated carbon is dispersed. A concentration of the precious metal compound is usually 0.1 wt % or more, preferably 0.2 wt % or more, especially preferably 0.5 wt % or more. Moreover, an upper limit of the concentration of the precious metal compound is usually 20 wt % or less, preferably 10 wt % or less, especially preferably 7 wt % or less. The amount of the activated carbon to be dispersed in the solution is appropriately set in such a manner that the amount of loading of the precious metal with respect to the activated carbon before the loading indicates a desired value in the finally obtained catalyst. Next, the reducing agent is added to the dispersion to reduce the precious metal in the precious metal compound, and the activated carbon on which the reduced precious metal is supported can be obtained.

The reducing agent which can be used in the present invention is not especially limited. However, examples thereof include hydrazine, formalin, sodium boron hydride, hydrogen, formic acid, salts of formic acid, ethylene, propylene and isobutylene.

Since reduction temperature and reduction time differ with the reduction method, the precious metal compound for use, the solvent, the reducing agent, and the like, it is not necessarily appropriate define, but in the liquid-phase reduction the reduction temperature is usually 0 to 100° C. and the reduction time is 0.5 to 24 hours.

After the reduction, the activated carbon on which the precious metal is supported (hereinafter referred to as the catalyst) is separated from the dispersion liquid. This method for separation is not especially limited, but methods such as filtration, and centrifugation can be used. The separated catalyst is appropriately dried. A method for drying is not especially limited, and various methods can be used.

It is to be noted that the concentration of a precious metal element contained in the solution separated from the catalyst after the reduction is preferably set to 10 mg/l or less. This amount can be controlled by concentration of the precious metal compound before the reduction or reduction conditions. The presence of the precious metal element in the solution can easily be confirmed by addition of the reducing agents such as hydrazine. The content of the precious metal element in the solution can be quantitatively determined by elemental analysis such as ICP.

As described above, the catalyst for the preparation of the α,β-unsaturated carboxylic acid of the present invention can be prepared.

The catalyst may also be activated before subjected to the liquid-phase oxidation. A method of activation is not especially limited, and, for example, a method in which the catalyst is heated under a reductive atmosphere in a hydrogen flow is general.

Next, the preparation method of the α,β-unsaturated carboxylic acid will be described in which the olefin or α,β-unsaturated aldehyde is oxidized with molecular oxygen in the liquid phase by the use of the catalyst for the preparation of the α,β-unsaturated carboxylic acid of the present invention.

Examples of the olefin which is the raw material of liquid-phase oxidation include propylene, isobutylene, 1-butene and 2-butene. Examples of the α,β-unsaturated aldehyde which is the raw material include acrolein, methacrolein, crotonaldehyde (β-methyl acrolein) and cinnam aldehyde (β-phenyl acrolein).

When the raw material is the olefin, the α,β-unsaturated carboxylic acid to be prepared in the liquid-phase oxidation is an α,β-unsaturated carboxylic acid having the same carbon skeleton as that of the olefin. When the raw material is the α,β-unsaturated aldehyde, an aldehyde group of the α,β-unsaturated aldehyde is changed to a carboxyl group in the α,β-unsaturated carboxylic acid.

The catalyst of the present invention is suitable for the liquid-phase oxidation in which the acrylic acid is prepared from propylene or acrolein, or the methacrylic acid is prepared from isobutylene or methacrolein.

The olefin or α,β-unsaturated aldehyde which is the raw material may contain a small amount of saturated hydrocarbons and/or lower saturated aldehydes as impurities.

A source of molecular oxygen for use in the reaction is economically preferably air, but pure oxygen or a mixed gas of pure oxygen and air may also be used. If necessary, it is also possible to use a mixed gas obtained by dilution of air or pure oxygen with nitrogen, carbon dioxide, steam, or the like.

The solvent for use in the liquid-phase oxidation is not especially limited, and it is possible to use water; alcohols such as tertiary butanol and cyclohexanol; ketones such as acetone, methyl ethyl ketone, and methyl isobutyl ketone; organic acids such as acetic acid, propionic acid, n-butyric acid, iso-butyric acid, n-valeric acid, and iso-valeric acid; esters of organic acid such as ethyl acetate and methyl propionate; hydrocarbons such as hexane, cyclohexane, and toluene; or a mixed solvent of two or more selected from these groups. Above all, the mixed solvent of one or two or more solvents selected from a group consisting of alcohols, ketones, organic acids, and esters of organic acid, and water is preferable. The amount of water in the water-containing mixed solvent is not especially limited, but the lower limit is preferably 2 wt % or more, more preferably 5 wt % or more with respect to the mass of the mixed solvent. The upper limit of the amount of water is preferably 70 wt % or less, more preferably 50 wt % or less. The solvent preferably has a homogeneous state, but may also have a heterogeneous state.

The liquid-phase oxidation may also be carried out in either of a continuous system and batch system, and the continuous system is preferable in consideration of productivity.

The amount of the olefin or α,β-unsaturated aldehyde which is the raw material is usually 0.1 part by weight or more, preferably 0.5 part by weight or more with respect to 100 parts by weight of the solvent. The upper limit of the amount of the raw material is usually 20 parts by weight or less, preferably 10 parts by weight or less.

The amount of molecular oxygen is usually 0.1 mol or more, preferably 0.3 mol or more, more preferably 0.5 mol or more with respect to 1 mol of the olefin or α,β-unsaturated aldehyde which is the raw material. The upper limit of the molecular oxygen amount is usually 20 mol or less, preferably 15 mol or less, more preferably 10 mol or less.

The catalyst is usually used in a suspended state in the reaction mixture, and may also be used in a fixed bed. The amount of the catalyst existing in a reaction unit is usually 0.1 part by weight or more, preferably 0.5 part by weight or more, especially preferably 1 part by weight or more with respect to 100 parts by weight of the solution existing in the reaction unit. The upper limit of the usage of the catalyst is usually 30 parts by weight or less, more preferably 20 parts by weight or less, especially preferably 15 parts by weight or less.

A reaction temperature and reaction pressure are appropriately selected depending on the solvent and raw material for use. The lower limit of the reaction temperature is usually 30° C. or more, preferably 50° C. or more, and the upper limit is usually 200° C. or less, preferably 150° C. or less. The lower limit of the reaction pressure is usually an atmospheric pressure (0 MPa) (gauge pressure) or more, preferably 0.5 MPa (gauge pressure) or more. The upper limit is usually 10 MPa (gauge pressure) or less, preferably 5 MPa (gauge pressure) or less.

### EXAMPLES

The present invention will hereinafter be described more concretely with examples and comparative examples, but the present invention is not limited to the examples. In the following examples and comparative examples, “parts” mean “parts by weight”.

(Analysis of Raw Material and Products)

The raw materials and products were analyzed using gas chromatography. Conversion of an olefin or α,β-unsaturated aldehyde, selectivity of the α,β-unsaturated aldehyde to be prepared, selectivity of a polymer/oligomer to be prepared, and selectivity and yield of the α,β-unsaturated carboxylic acid to be prepared are defined as follows:

$$Conversion ( \% ) of the olefin or α , β \text{-} unsaturated aldehyde = ( B / A ) \times 100 ;$$

$$Selectivity ( \% ) of the α , β \text{-} unsaturated aldehyde = ( C / B ) \times 100 ;$$

$$Selectivity ( \% ) of the α , β \text{-} unsaturated carboxylic acid = ( D / B ) \times 100 ;$$

$$Selectivity ( \% ) of the polymer / oligomer = ( E / B ) \times 100 ; and$$

$$Yield ( \% ) of the α , β \text{-} unsaturated carboxylic acid = ( D / A ) \times 100 ,$$

where A denotes a mol number of the supplied olefin or α,β-unsaturated aldehyde, B denotes a mol number of the reacted olefin or α,β-unsaturated aldehyde, C denotes a mol number of the prepared α,β-unsaturated aldehyde, D denotes a mol number of the prepared α,β-unsaturated carboxylic acid, and E denotes a mol number of the polymer and oligomer, which is the mol number of the olefin or α,β-unsaturated aldehyde unit, in which a total weight (unit: g) of the prepared polymer and oligomer is divided by a molecular weight of the supplied olefin or α,β-unsaturated aldehyde. Here, in the oxidation reaction of the α,β-unsaturated aldehyde, C/B=0.

### Example 1

(Preparation of Catalyst)

An activated carbon powder having a specific surface area of 700 m²/g prepared from coal was selected as a carrier from various types of activated carbons.

500 parts of acetic acid and 2.5 parts of palladium acetate were placed in an autoclave, and after heating and making a solution at 80° C., 24.0 parts of the above activated carbon were added thereto. Then, the mixture was stirred at 80° C. for one hour in a state in which the autoclave was opened. The autoclave was closed, and a gaseous phase in the autoclave was replaced with nitrogen while a liquid phase was stirred. Propylene was introduced into the autoclave to an inner pressure of 0.6 MPa (gauge pressure), followed by stirring at 80° C. for one hour.

Thereafter, the stirring was stopped, the pressure in the autoclave was released, and thereafter a reaction mixture was taken out. The resultant precipitate was collected from the reaction mixture under a nitrogen flow by filtration. At this time, a small amount of hydrazine monohydrate was added to a filtrate, whereby it was confirmed that any deposit of palladium was not observed.

The obtained precipitate was dried overnight at 100° C. under the nitrogen flow to obtain a catalyst in which the palladium metal was supported. The amount of loading of the palladium metal in the catalyst was 5 wt %.

(Reaction Evaluation)

In the autoclave including a stirring device (hereinafter referred to as the reaction unit), 70 parts of a 75 wt % aqueous acetic acid solution were charged as a reaction solvent, 5.5 parts of the above catalyst and 2.5 parts of methacrolein were added, and the reaction unit was closed. Subsequently, the stirring was started, and the temperature was raised to 90° C. After introducing nitrogen into the reaction unit to an inner pressure of 1.0 MPa (gauge pressure), air was introduced to an inner pressure of 3.5 MPa (gauge pressure). In this state, oxidation reaction of methacrolein was carried out for 20 minutes.

After the end of the reaction, the contents of the reaction unit were cooled to 20° C. by an ice bath. A gas sampling bag was attached to a gas outlet of the reaction unit, a valve of the gas outlet was opened to collect an exhausted gas, and the pressure in the reaction unit was released. The reaction mixture containing the catalyst was taken out of the reaction unit, the catalyst was separated by the centrifugation, and only the reaction solution was collected.

As a result, the conversion of methacrolein was 85.7%, the selectivity of methacrylic acid was 76.6%, the selectivity of a polymer/oligomer was 13.6%, and the yield of methacrylic acid was 65.6%.

### Example 2

A catalyst was prepared and the reaction was evaluated in the same manner as in Example 1 except that the carrier was changed to an activated carbon powder having a specific surface area of 590 m²/g prepared from coal.

As a result, the conversion of methacrolein was 93.2%, the selectivity of methacrylic acid was 74.9%, the selectivity of a polymer/oligomer was 14.9%, and the yield of methacrylic acid was 69.8%.

### Example 3

A catalyst was prepared and the reaction was evaluated in the same manner as in Example 1 except that the carrier was changed to an activated carbon powder having a specific surface area of 850 m²/g prepared from coconut shells.

As a result, the conversion of methacrolein was 94.9%, the selectivity of methacrylic acid was 71.9%, the selectivity of a polymer/oligomer was 16.3%, and the yield of methacrylic acid was 68.2%.

### Example 4

A catalyst was prepared and the reaction was evaluated in the same manner as in Example 1 except that the carrier was changed to an activated carbon powder having a specific surface area of 1200 m²/g prepared from coconut shells.

As a result, the conversion of methacrolein was 62.0%, the selectivity of methacrylic acid was 55.6%, the selectivity of a polymer/oligomer was 26.3%, and the yield of methacrylic acid was 34.5%.

### Comparative Example 1

A catalyst was prepared and the reaction was evaluated in the same manner as in Example 1 except that the carrier was changed to an activated carbon powder having a specific surface area of 1400 m²/g prepared from coal.

As a result, the conversion of methacrolein was 39.6%, the selectivity of methacrylic acid was 16.6%, the selectivity of a polymer/oligomer was 67.5%, and the yield of methacrylic acid was 6.6%.

### Comparative Example 2

A catalyst was prepared and the reaction was evaluated in the same manner as in Example 1 except that the carrier was changed to an activated carbon powder having a specific surface area of 1600 m²/g prepared from coconut shells.

As a result, the conversion of methacrolein was 15.1%, the selectivity of methacrylic acid was 52.5%, the selectivity of a polymer/oligomer was 36.7%, and the yield of methacrylic acid was 7.9%.

### Example 5

In the reaction unit, 120 parts of a 75 wt % aqueous tertiary butanol solution were charged as a reaction solvent, 10.0 parts of the catalyst prepared in Example 1 were added, and the reaction unit was closed. Subsequently, 6.6 parts of liquefied isobutylene was introduced into the reaction unit, the stirring was started, and the temperature was raised to 90° C. Air was introduced into the reaction unit to an inner pressure of 3.5 MPa (gauge pressure). In this state, the oxidation reaction of isobutylene was carried out for 40 minutes.

After the end of the reaction, the contents of the reaction unit were cooled to 20° C. by the ice bath. The gas sampling bag was attached to the gas outlet of the reaction unit, the valve of the gas outlet was opened to collect an exhausted gas, and the pressure in the reaction unit was released. The reaction mixture containing the catalyst was taken out of the reaction unit, the catalyst was separated by the centrifugation, and only the reaction solution was collected.

As a result, the conversion of isobutylene was 36.2%, the selectivity of methacrolein was 40.2%, the selectivity of methacrylic acid was 11.1%, the selectivity of a polymer/oligomer was 35.2%, and the yield of methacrylic acid was 4.0%.

### Comparative Example 3

The reaction was evaluated in the same manner as in Example 4 except that the catalyst was changed to the catalyst prepared in Comparative Example 1.

As a result, the conversion of isobutylene was 16.7%, the selectivity of methacrolein was 50.6%, the selectivity of methacrylic acid was 7.2%, the selectivity of a polymer/oligomer was 29.3%, and the yield of methacrylic acid was 1.2%.

The above-described results are shown in Tables 1 and 2.

TABLE 1

| | Raw material | Specific surface | | Selectivity of | Selectivity of | Yield of |
|---|---|---|---|---|---|---|
| | of activated | area of activated | Conversion of | methacrylic | polymer/oligomer | methacrylic |
| | carbon | carbon (m²/g) | methacrolein (%) | acid (%) | (%) | acid (%) |
| Ex. 1 | Coal | 700 | 85.7 | 76.6 | 13.6 | 65.6 |
| Ex. 2 | Coal | 590 | 93.2 | 74.9 | 14.9 | 69.8 |
| Ex. 3 | Coconut | 850 | 94.9 | 71.9 | 16.3 | 68.2 |
| | shells | | | | | |
| Ex. 4 | Coconut | 1200 | 62.0 | 55.6 | 26.3 | 34.5 |
| | shells | | | | | |
| Comp. | Coal | 1400 | 39.6 | 16.6 | 67.5 | 6.6 |
| Ex. 1 | | | | | | |
| Comp. | Coconut | 1600 | 15.1 | 52.5 | 36.7 | 7.9 |
| Ex. 2 | shells | | | | | |

TABLE 2

| | Raw material | Specific surface | | | Selectivity of | Selectivity of | Yield of |
|---|---|---|---|---|---|---|---|
| | of activated | area of activated | Conversion of | Selectivity of | methacrylic | polymer/oligomer | methacrylic |
| | carbon | carbon (m²/g) | isobutylene (%) | methacrolein (%) | acid (%) | (%) | acid (%) |
| Ex. 5 | Coal | 700 | 36.2 | 40.2 | 11.1 | 35.2 | 4.0 |
| Comp. | Coal | 1400 | 16.7 | 50.6 | 7.2 | 29.3 | 1.2 |
| Ex. 3 | | | | | | | |

In this manner, it has been revealed that an α,β-unsaturated carboxylic acid can be prepared in a high yield from an olefin or α,β-unsaturated aldehyde by the use of the catalyst for the preparation of the α,β-unsaturated carboxylic acid of the present invention.

### INDUSTRIAL APPLICABILITY

In accordance with a preparation method of a catalyst for preparation of an α,β-unsaturated carboxylic acid of the present invention, a catalyst for oxidizing an olefin or α,β-unsaturated aldehyde with molecular oxygen in a liquid phase to prepare an α,β-unsaturated carboxylic acid in a high yield can be prepared. By the use of this catalyst, the α,β-unsaturated carboxylic acid can be prepared with the high yield.

1-12. (canceled)

13. A catalyst for preparation of an α,β-unsaturated carboxylic acid by oxidizing an olefin or α,β-unsaturated aldehyde with molecular oxygen in a liquid phase, comprising a precious metal supported on activated carbon having a specific surface area of 100 m²/g or more and 1300 m²/g or less.

14. The catalyst for preparation of an α,β-unsaturated carboxylic acid according to claim 13, wherein the precious metal is one or more selected from a group consisting of palladium, platinum, rhodium, ruthenium, iridium, gold, silver, and osmium.

15. The catalyst for preparation of an α,β-unsaturated carboxylic acid according to claim 13, wherein an amount of loading of the precious metal is in a range of 0.1 to 40 wt % with respect to the activated carbon before loading.

16. The catalyst for preparation of an α,β-unsaturated carboxylic acid according to claim 13, which is a catalyst for preparation of acrylic acid from propylene or acrolein, or a catalyst for preparation of methacrylic acid from isobutylene or methacrolein.

17. The catalyst for preparation of an α,β-unsaturated carboxylic acid according to claim 13, wherein the specific surface area of the activated carbon is 100 m²/g or more and 1000 m²/g or less.

18. The catalyst for preparation of an α,β-unsaturated carboxylic acid according to claim 17, wherein the precious metal is one or more selected from the group consisting of palladium, platinum, rhodium, ruthenium, iridium, gold, silver, and osmium.

19. The catalyst for preparation of an α,β-unsaturated carboxylic acid according to claim 17, wherein an amount of loading of the precious metal is in a range of 0.1 to 40 wt % with respect to the activated carbon before loading.

20. The catalyst for preparation of an α,β-unsaturated carboxylic acid according to claim 17, which is a catalyst for preparation of acrylic acid from propylene or acrolein, or a catalyst for preparation of methacrylic acid from isobutylene or methacrolein.

21. A preparation method of the catalyst for preparation of an α,β-unsaturated carboxylic acid according to claim 13, comprising selecting activated carbon having a specific surface area of 100 m²/g or more and 1300 m²/g or less and loading the precious metal on the activated carbon.

22. The preparation method of the catalyst for preparation of an α,β-unsaturated carboxylic acid according to claim 21, comprising reducing a precious metal compound corresponding to the precious metal to be loaded on the activated carbon with a reducing agent in the presence of the activated carbon.

23. The preparation method of the catalyst for preparation of an α,β-unsaturated carboxylic acid according to claim 22, comprising adding the reducing agent to a solution of the precious metal compound in which the activated carbon is dispersed, to reduce the precious metal compound, wherein the precious metal is loaded on the activated carbon.

24. The preparation method of the catalyst for preparation of an α,β-unsaturated carboxylic acid according to claim 23, wherein the precious metal compound is a chloride, oxide, acetate, nitrate, sulfate, tetra-ammine complex or acetylacetonate complex of the precious metal.

25. The preparation method of the catalyst for preparation of an α,β-unsaturated carboxylic acid according to claim 23, wherein a concentration of the precious metal compound in the solution is in a range of 0.1 to 20 wt %.

26. A preparation method of the catalyst for preparation of an α,β-unsaturated carboxylic acid according to claim 17, comprising selecting activated carbon having a specific surface area of 100 m²/g or more and 1000 m²/g or less and loading the precious metal on the activated carbon.

27. The preparation method of the catalyst for preparation of an α,β-unsaturated carboxylic acid according to claim 26, comprising reducing a precious metal compound corresponding to the precious metal to be loaded on the activated carbon with a reducing agent in the presence of the activated carbon.

28. The preparation method of the catalyst for preparation of an α,β-unsaturated carboxylic acid according to claim 27, comprising adding the reducing agent to a solution of the precious metal compound in which the activated carbon is dispersed, to reduce the precious metal compound, wherein the precious metal is loaded on the activated carbon.

29. The preparation method of the catalyst for preparation of an α,β-unsaturated carboxylic acid according to claim 28, wherein the precious metal compound is a chloride, oxide, acetate, nitrate, sulfate, tetra-ammine complex or acetylacetonate complex of the precious metal.

30. The preparation method of the catalyst for preparation of an α,β-unsaturated carboxylic acid according to claim 28, wherein a concentration of the precious metal compound in the solution is in a range of 0.1 to 20 wt %.

31. A preparation method of an α,β-unsaturated carboxylic acid, comprising oxidizing an olefin or α,β-unsaturated aldehyde with molecular oxygen in a liquid phase in the presence of the catalyst for preparation of an α,β-unsaturated carboxylic acid according to claim 13 to prepare the α,β-unsaturated carboxylic acid.

32. A preparation method of an α,β-unsaturated carboxylic acid, comprising oxidizing an olefin or α,β-unsaturated aldehyde with molecular oxygen in a liquid phase in the presence of the catalyst for preparation of an α,β-unsaturated carboxylic acid according to claim 17 to prepare the α,β-unsaturated carboxylic acid.
